(19) United States
(12) Patent Application Publication
(10) Pub. No.: US 2025/0390281 A1
(43) Pub. Date: Dec. 25, 2025

(54) SYSTEMS, METHODS, AND MEDIA FOR FACILITATING MEDICAL DEVICE SOFTWARE DEVELOPMENT COMPLIANCE

(71) Applicants: Michael Madera, Weeki Wachee, FL (US); Jonathan Werner, Lutz, FL (US)

(72) Inventors: Michael Madera, Weeki Wachee, FL (US); Jonathan Werner, Lutz, FL (US)

(21) Appl. No.: 18/748,890

(22) Filed: Jun. 20, 2024

(51) Int. Cl. G06F 8/20; G06F 8/73; G06F 11/36; G16H 40/20

(52) U.S. Cl. CPC G06F 8/22; G06F 8/73; G06F 11/3692; G16H 40/20

(57) ABSTRACT

Systems, methods, and computer-readable media for facilitating medical device software development compliance are disclosed. The systems, methods, and media can receive role assignment data identifying a first plurality of user accounts with assigned roles; receive a request to generate a first project profile for developing a first medical device software application; receive a request to generate first documentation associated with the medical device software application; receive first input data in response to presenting one or more first questions; determine a software safety classification of the first medical device software application; generate the first documentation; request approval of the first documentation from each of the first plurality of user accounts with assigned roles; receive the approval of the first documentation from each of the first plurality of user accounts; generate a plurality of project phases associated with the first project profile based at least on the software safety classification.

### FIELD OF DISCLOSURE

The present disclosure generally relates to systems, methods, and media for facilitating medical device software development compliance.

### BACKGROUND

In general, developing medical device software can involve the collaboration between multiple users that create various requirements for the medical device software. However, original requirements can change, and additional requirements can be created at different times during software development. Ensuring that all requirements are tracked during software development can become increasingly complex for software with increasing functionality.

There is a need for methods, systems, and media for facilitating medical device software development compliance that can make the collaboration between multiple users more efficient, so that requirements are better tracked and satisfied as the medical device software is developed.

### SUMMARY

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

In some embodiments, a method for facilitating medical device software development compliance is disclosed, the method comprising receiving role assignment data identifying a first plurality of user accounts with assigned roles; receiving a request to generate a first project profile for developing a first medical device software application; receiving a request to generate first documentation associated with the medical device software application; receiving first input data in response to presenting one or more first questions; determining a software safety classification of the first medical device software application based on the first input data; receiving second input data indicating one or more first risk controls associated with the medical device software application; in response to receiving the first input data and the second input data, generating the first documentation including the software safety classification and the first risk controls; requesting approval of the first documentation from each of the first plurality of user accounts with assigned roles; receiving the approval of the first documentation from each of the first plurality of user accounts with assigned roles; modifying the first documentation to indicate the approval from each of the first plurality of user accounts and their assigned roles; storing the first documentation in memory; including at least a first identifier of the first documentation in a document management dashboard associated with the first project profile of the medical device software application; generating one or more first approved requirements based on the one or more first risk controls, wherein a first approved requirement is associated with a first requirement identifier; generating a plurality of project phases associated with the first project profile based at least on the software safety classification, wherein: the plurality of project phases are associated with respective sets of tasks; one or more tasks of the sets of tasks are generated based at least on the software safety classification; at least a first task of the one or more tasks is associated with the one or more first approved requirements.

In some embodiments, the method for facilitating medical device software development compliance can include any method, process, subprocess, or any combination thereof, disclosed herein.

In some embodiments, a system for facilitating medical device software development compliance is disclosed, the system comprising memory; and one or more processors operably coupled to the memory, wherein the one or more processors are configured at least to: receive role assignment data identifying a first plurality of user accounts with assigned roles; receive a request to generate a first project profile for developing a first medical device software application; receive a request to generate first documentation associated with the medical device software application; receive first input data in response to presenting one or more first questions; determine a software safety classification of the first medical device software application based on the first input data; receive second input data indicating one or more first risk controls associated with the medical device software application; in response to receiving the first input data and the second input data, generate the first documentation including the software safety classification and the first risk controls; request approval of the first documentation from each of the first plurality of user accounts with assigned roles; receive the approval of the first documentation from each of the first plurality of user accounts with assigned roles; modifying the first documentation to indicate the approval from each of the first plurality of user accounts and their assigned roles; store the first documentation in memory; include at least a first identifier of the first documentation in a document management dashboard associated with the first project profile of the medical device software application; generate one or more first approved requirements based on the one or more first risk controls, wherein a first approved requirement is associated with a first requirement identifier; generate a plurality of project phases associated with the first project profile based at least on the software safety classification, wherein: the plurality of project phases are associated with respective sets of tasks; one or more tasks of the sets of tasks are generated based at least on the software safety classification; at least a first task of the one or more tasks is associated with the one or more first approved requirements.

In some embodiments, the one or more processors can be configured to perform any method, process, subprocess, or any combination thereof, disclosed herein.

In some embodiments, a non-transitory computer-readable medium is disclosed, comprising instructions, that when executed by one or more processors, cause the one or more processors to perform a method for facilitating medical device software development compliance, the method comprising receiving role assignment data identifying a first plurality of user accounts with assigned roles; receiving a request to generate a first project profile for developing a first medical device software application; receiving a request to generate first documentation associated with the medical device software application; receiving first input data in response to presenting one or more first questions; determining a software safety classification of the first medical device software application based on the first input data; receiving second input data indicating one or more first risk controls associated with the medical device software application; in response to receiving the first input data and the second input data, generating the first documentation including the software safety classification and the first risk controls; requesting approval of the first documentation from each of the first plurality of user accounts with assigned roles; receiving the approval of the first documentation from each of the first plurality of user accounts with assigned roles; modifying the first documentation to indicate the approval from each of the first plurality of user accounts and their assigned roles; storing the first documentation in memory; including at least a first identifier of the first documentation in a document management dashboard associated with the first project profile of the medical device software application; generating one or more first approved requirements based on the one or more first risk controls, wherein a first approved requirement is associated with a first requirement identifier; generating a plurality of project phases associated with the first project profile based at least on the software safety classification, wherein: the plurality of project phases are associated with respective sets of tasks; one or more tasks of the sets of tasks are generated based at least on the software safety classification; at least a first task of the one or more tasks is associated with the one or more first approved requirements.

In some embodiments, the one or more processors can perform any method, process, subprocess, or any combination thereof, disclosed herein.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings may contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 illustrates a block diagram of a system for facilitating medical device software development compliance, according to some embodiments disclosed herein;

FIG. 2 illustrates a block diagram of hardware of a computing device for facilitating medical device software development compliance, according to some embodiments disclosed herein;

FIG. 3A illustrates a flowchart of a method for facilitating medical device software development compliance, according to some embodiments disclosed herein;

FIG. 3B illustrates a flowchart of a method for facilitating medical device software development compliance, according to some embodiments disclosed herein; and

FIGS. 4A-4AH illustrate user interfaces generated at a computing device, according to some embodiments disclosed herein.

The drawings are not necessarily to scale, and certain features and certain views of the drawings may be shown exaggerated in scale or in schematic in the interest of clarity and conciseness.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more other embodiments, it is to be understood that the embodiments disclosed herein are illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof.

Any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Furthermore, it is important to note that, as used herein, “a” and “an” each generally mean “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims.

The present disclosure provides systems, methods, and media for facilitating medical device software development compliance.

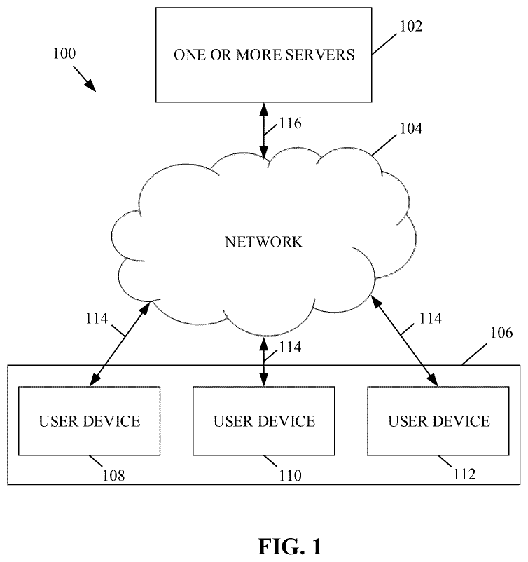

Referring to FIG. 1, a system 100 for facilitating medical device software development compliance can be used with some embodiments disclosed herein. In some embodiments, system 100 can comprise one or more servers 102, a network 104 (e.g., communication network), one or more user devices 106, or any combination thereof. In some embodiments, the one or more user devices 106 can include a first user device 108, a second user device 110, a third user device 112, any other user device(s), or any combination thereof.

The one or more servers 102, the one or more user devices 106, or any combination thereof can include any suitable computing devices for storing data, programs, or a combination thereof, for facilitating medical device software development compliance. In some embodiments, the one or more servers 102, the one or more user devices 106, or any combination thereof can store any suitable data about one or more medical device software applications. For example, the data about the one or more medical device software applications can include data about any user accounts (e.g., authentication information such as username and password), project profiles, questions, software safety classifications, risk controls, approvals from associated user accounts, documentations, identifiers, project phases, tasks, software defects, defect tickets, requirements, a requirements traceability matrix, any other suitable data about one or more medical device software applications, or any combination thereof.

In some embodiments, the one or more servers 102, the one or more user devices 106, or any combination thereof can be configured to perform any method, any process, any subprocess, or any combination thereof, disclosed herein.

The network 104 can include a wired network, a wireless network, or a combination thereof. In some embodiments, the network 104 can include the Internet, an intranet, a wide-area network (WAN), a local-area network (LAN), a digital subscriber line (DSL) network, a frame relay network, an asynchronous transfer mode (ATM) network, a virtual private network (VPN), any other suitable communication network, or any combination thereof. In some embodiments, one or more communications links 114 can connect the one or more user devices 106 to the network 104. In some embodiments, one or more communication links 116 can connect the network 104 to the one or more servers 102. The one or more communication links 114, 116 can include any communication links suitable for communicating information between the one or more user devices 106 and the one or more servers 102, such as, for example, network links, dial-up links, wireless links, hard-wired links, any other suitable communications links, or any combination thereof.

In some embodiments, the one or more servers 102 can include one or more computing devices. While the one or more servers 102 are illustrated as one device, any suitable number of computing devices can be included in the one or more servers 102 in some embodiments.

In some embodiments, the one or more user devices 106 can include one or more computing devices. While three user devices 108, 110, 112 are illustrated, any suitable number of computing devices can be included in the one or more user devices 106 in some embodiments. In some embodiments, each user device 106 of the one or more user devices 106 can be associated with a respective user account of a plurality of user accounts.

In some embodiments, the one or more servers 102 and the one or more user devices 106 can be implemented using any suitable hardware. For example, any device of the one or more servers 102 and the one or more user devices 106 can be implemented using any suitable general-purpose computer or special-purpose computer. Any general-purpose computer or special-purpose computer can include any suitable hardware.

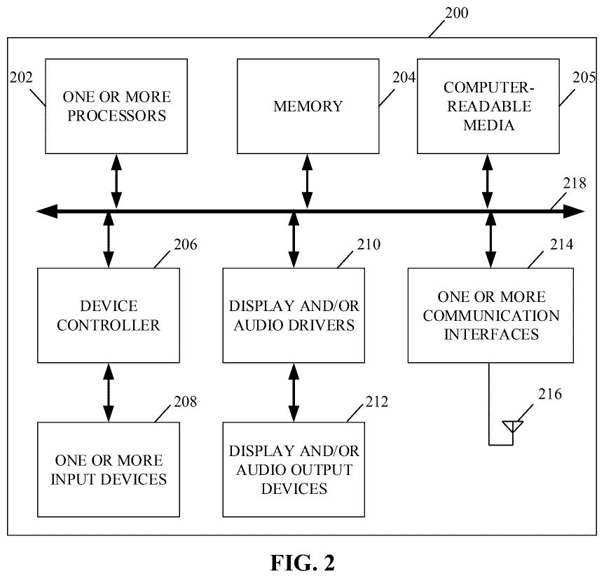

Referring to FIG. 2, an example hardware of a computing device 200 is illustrated. In some embodiments, the computing device 200 can include one or more processors 202, memory 204, a device controller 206, one or more input devices 208, display and/or audio drivers 210, display and/or audio output devices 212, one or more communication interfaces 214, one or more antennas 216, a bus 218, or any combination thereof.

In some embodiments, the one or more processors 202 can include any suitable hardware processor, such as a central processing unit (CPU), a graphics processing unit (GPU), a tensor processing unit (TPU), an accelerated processing unit (APU), any other type of processing unit, or any combination thereof. In some embodiments, the one or more processors 202 can include a microprocessor, a micro-controller, a digital signal processor, dedicated logic, an application-specific integrated circuit (ASIC), a field programmable gate array (FPGA), an accelerator (e.g., an artificial intelligence (AI) accelerator or a cryptographic accelerator), any other suitable circuitry for controlling the functioning of a general purpose computer or a special purpose computer, or any combination thereof.

In some embodiments, one or more processors 202 of any server of the one or more servers 102 can be controlled by a server program stored in memory 204 of the server. For example, in some embodiments, the server program can cause the one or more processors 202 to perform any method, process, subprocess, or any combination thereof.

In some embodiments, one or more processors 202 of any user device (e.g., 108, 110, 112 in FIG. 1) of the one or more user devices 106 can be controlled by a computer program stored in memory 204 of the user device. For example, the computer program can cause the one or more processors 202 to perform any method, process, subprocess, or any combination thereof, disclosed herein.

In some embodiments, the memory 204 can include any suitable memory, storage, or a combination thereof for storing programs, data, and/or any other suitable information. For example, memory 204 can include volatile memory, non-volatile memory, or any combination thereof. In some embodiments, memory 204 can include random access memory, read-only memory, flash memory, a hard disk drive, a solid state drive, optical media, any other suitable memory, or any combination thereof. In some embodiments, the memory 204 can include transitory or non-transitory computer-readable media for storing instructions that, when executed by one or more processors 202, cause the one or more processors 202 to perform any method, process, subprocess, or any combination thereof, disclosed herein.

In some embodiments, computer-readable media 205 can be operably coupled to the one or more processors 202. In some embodiments, the computer-readable media 205 can store instructions that, when executed by the one or more processors 202, cause the one or more processors 202 to perform any process or subprocess disclosed herein. In some embodiments, computer readable media 205 can be transitory or non-transitory. For example, non-transitory computer readable media can include media such as magnetic media (such as hard disks, floppy disks, and/or any other suitable magnetic media), optical media (such as compact discs, digital video discs, Blu-ray discs, and/or any other suitable optical media), semiconductor media (such as flash memory, electrically programmable read-only memory (EPROM), electrically erasable programmable read-only memory (EEPROM), and/or any other suitable semiconductor media), any suitable media that is not fleeting or devoid of any semblance of permanence during transmission, and/or any suitable tangible media. As another example, transitory computer readable media can include signals on networks, in wires, conductors, optical fibers, circuits, any suitable media that is fleeting and devoid of any semblance of permanence during transmission, and/or any suitable intangible media.

In some embodiments, the device controller 206 can include any suitable processor or circuitry for controlling and receiving any input from the one or more input devices 208. In some embodiments, the one or more input devices 208 can include a touchscreen, a keyboard, a mouse, one or more buttons, a voice recognition circuit, a camera, one or more sensors, a global positioning system (GPS) receiver, any other suitable input device, or any combination thereof. In some embodiments, the one or more sensors can include one or more accelerometers, one or more gyroscope sensors, one or more microphones, any other suitable sensors (e.g., an optical sensor, a temperature sensor, a near field sensor), or any combination thereof. In some embodiments, the one or more input devices 208 can be included in any device of the one or more servers 102, the one or more user devices 106, or any combination thereof.

In some embodiments, the display and/or audio drivers 210 can include any suitable circuitry for controlling and driving output to one or more display and/or audio output devices 212. For example, the output devices can include a display (e.g., including a touchscreen, a flat-panel display, a cathode ray tube display, a projector, any other suitable display or presentation device, or any combination thereof), one or more speakers, or a combination thereof.

In some embodiments, the one or more communication interfaces 214 can include any suitable circuitry for interfacing with one or more communication networks, such as network 104 as shown in FIG. 1. For example, the one or more communication interfaces 214 can include network interface card circuitry, wired communication circuitry, wireless communication circuitry, any other suitable communication network circuitry, or any combination thereof.

In some embodiments, the one or more antennas 216 can wirelessly communicate with a communication network (e.g., network 104). In some embodiments, the one or more antennas 216 can be omitted.

In some embodiments, the bus 218 can include any suitable communication system for communicating data, addresses, control signals, power, or any combination thereof, between two or more components 202, 204, 206, 210, and 214. In some embodiments, the bus 218 can include any suitable conductors that are constructed and arranged to communicate data, addresses, control signals, power, or any combination thereof, between two or more components 202, 204, 206, 210, and 214.

In some embodiments, any other suitable component(s) can be included in the computing device 200.

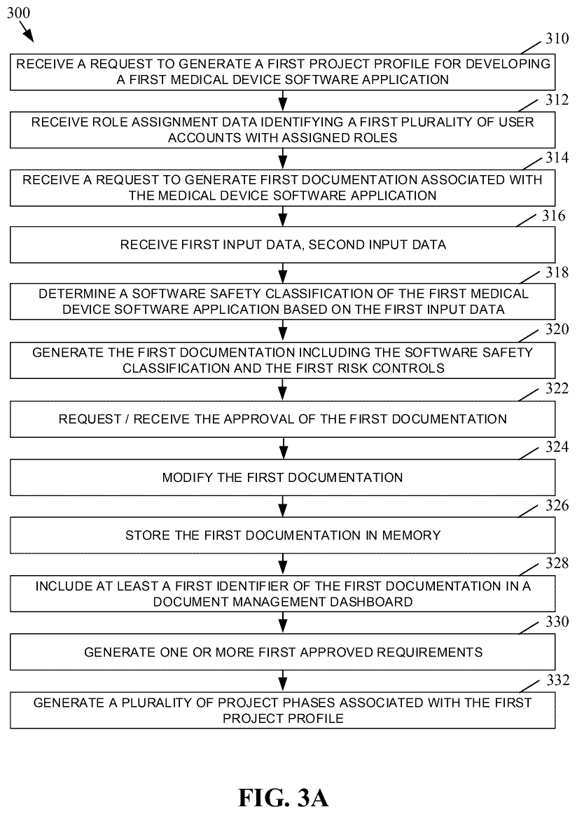

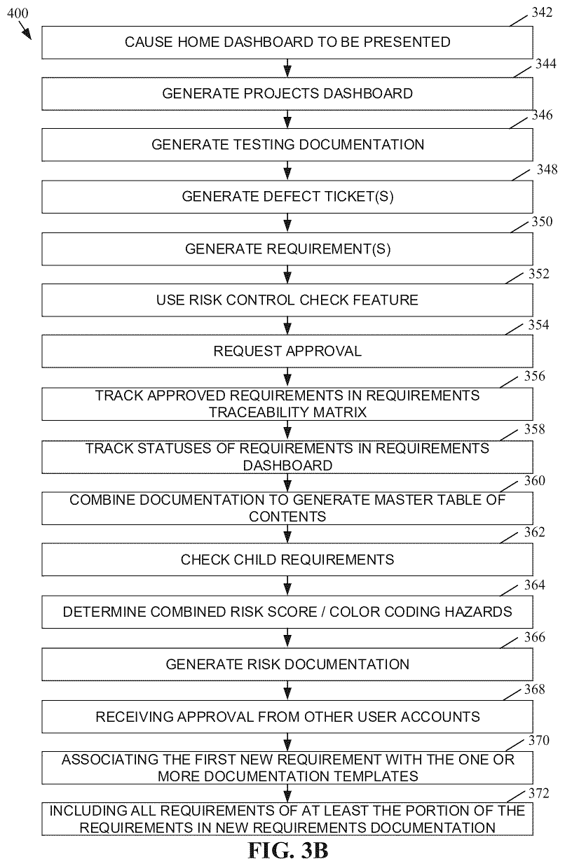

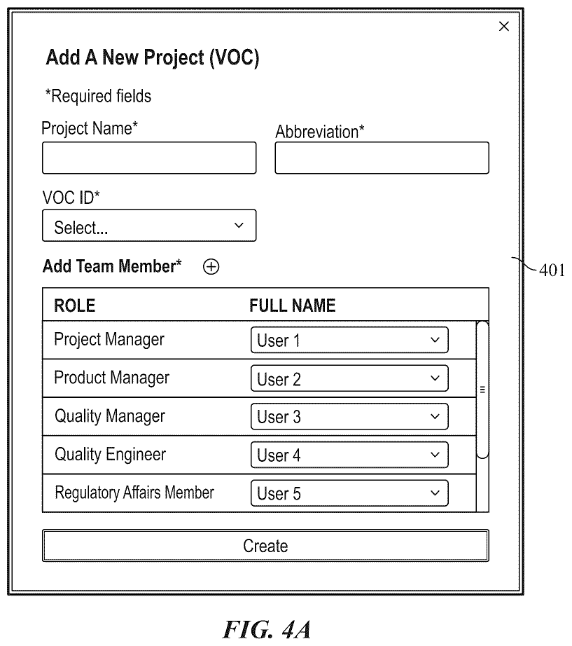

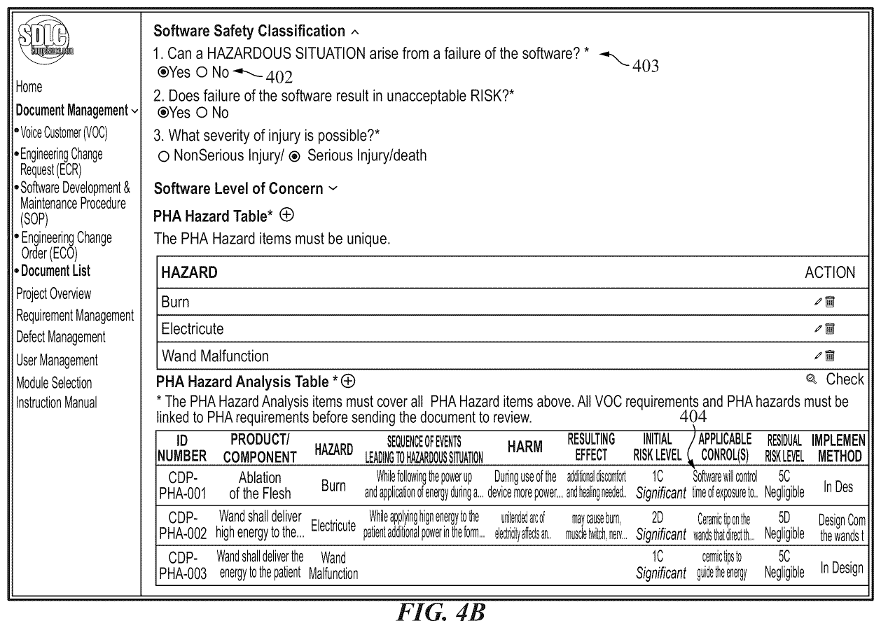

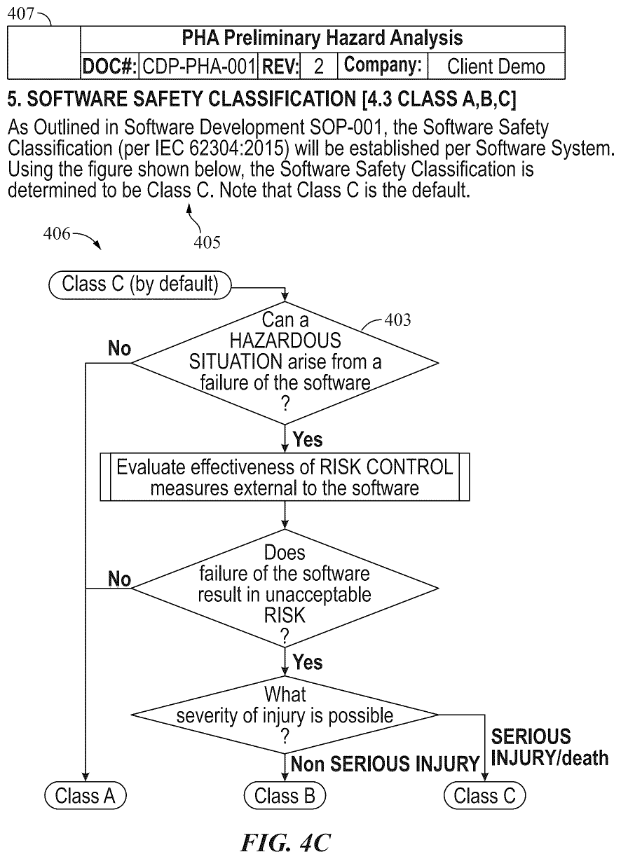

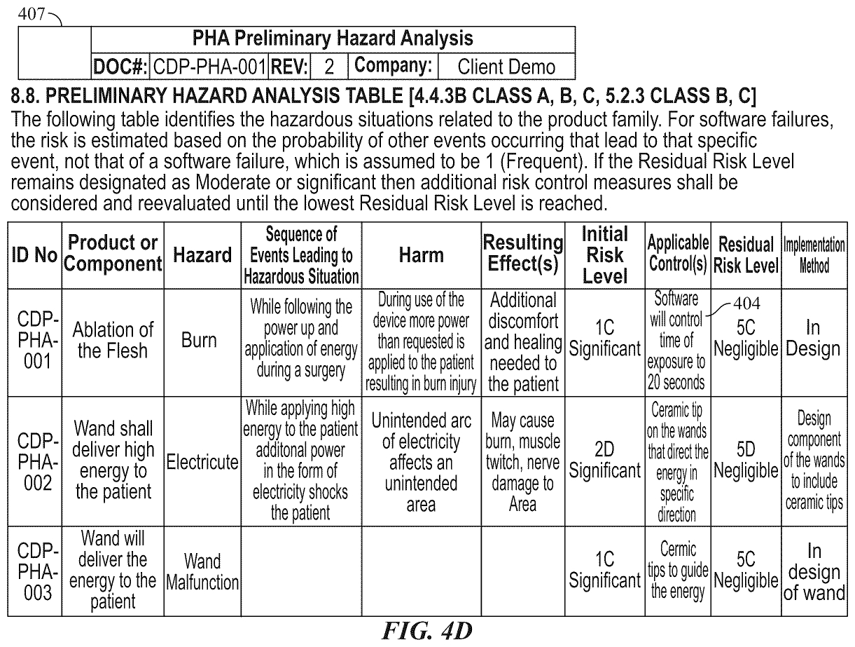

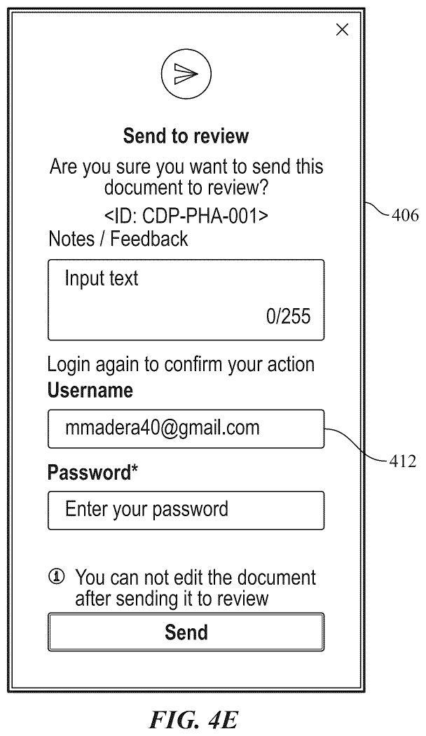

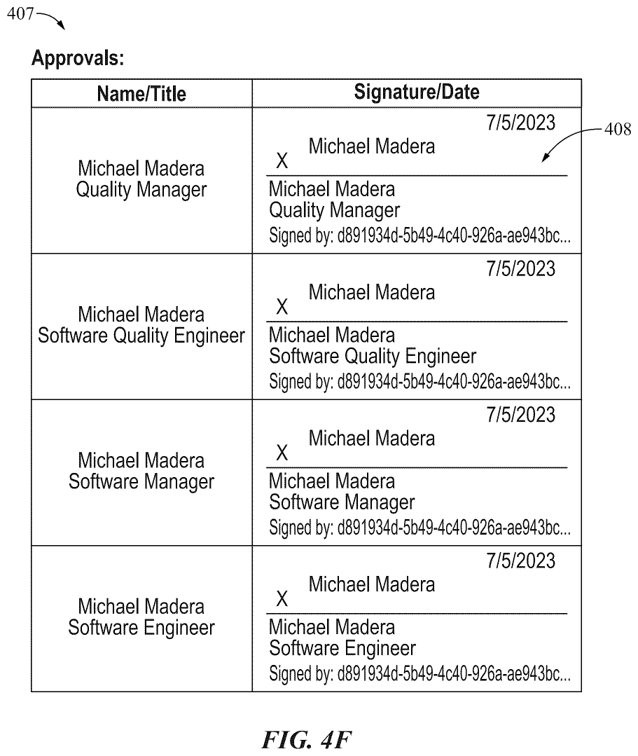

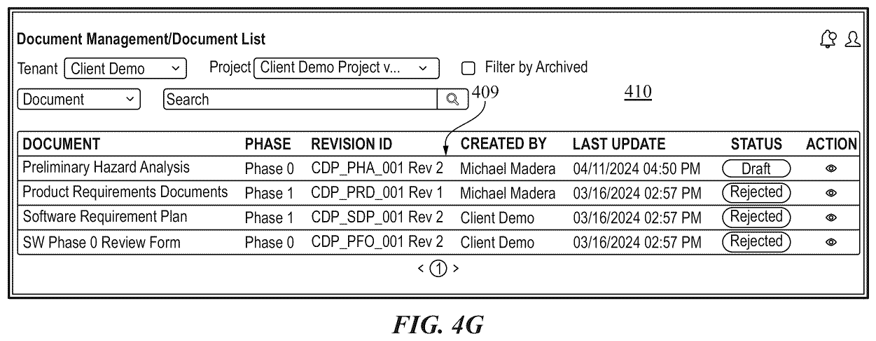

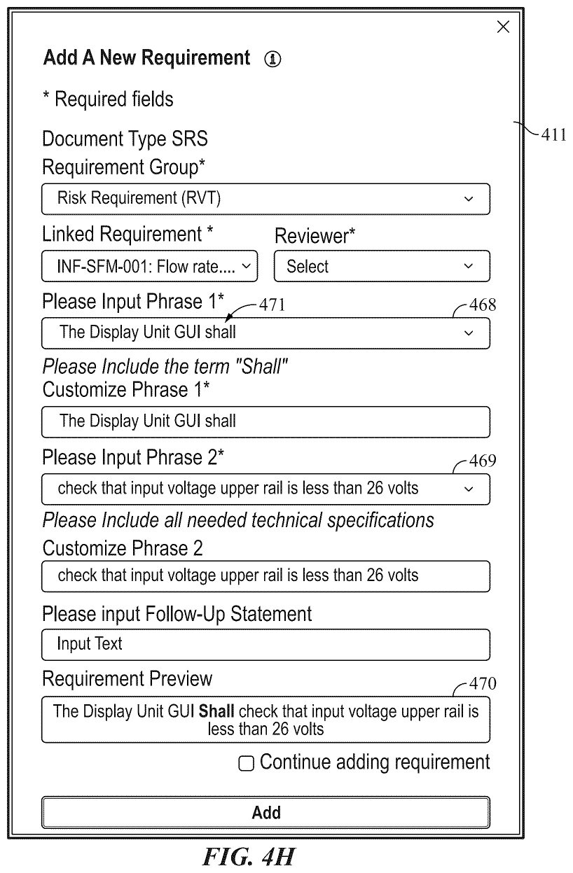

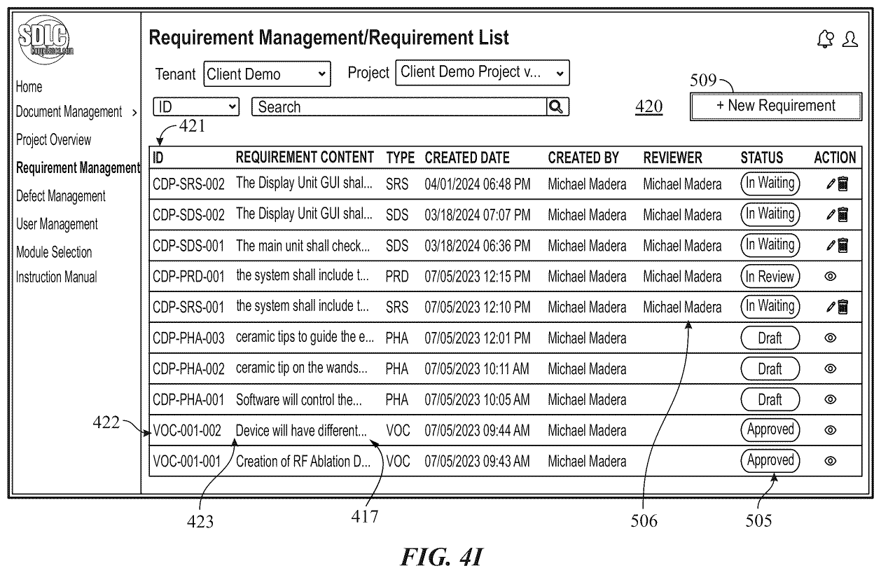

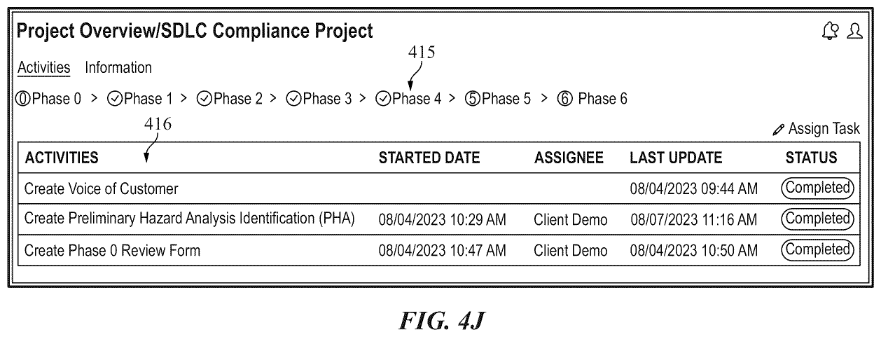

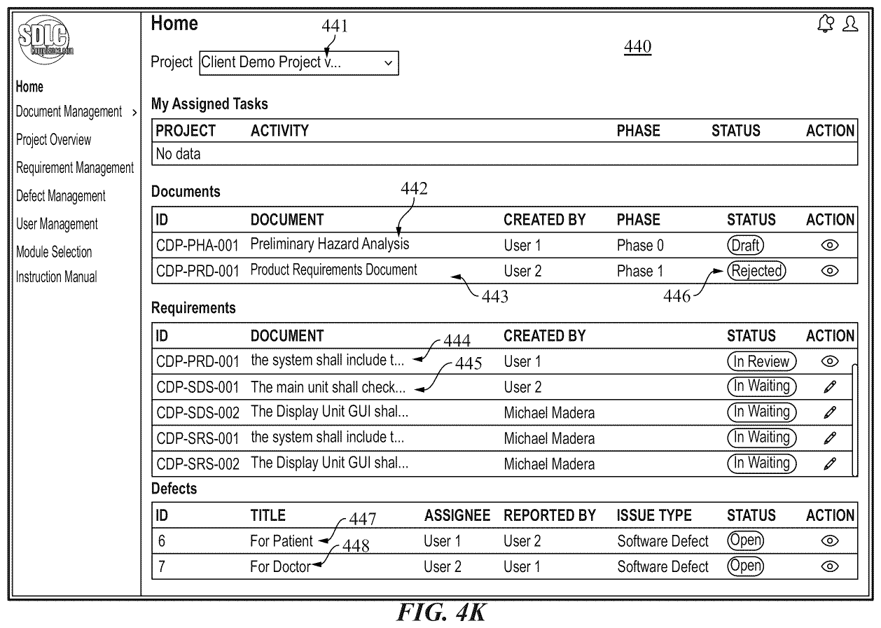

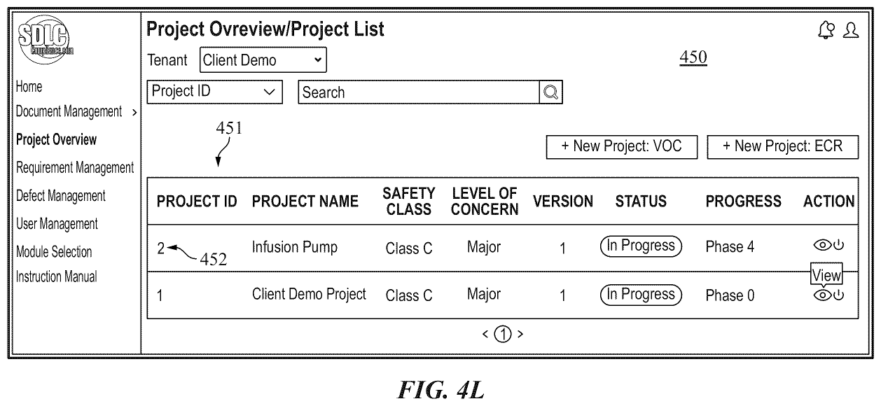

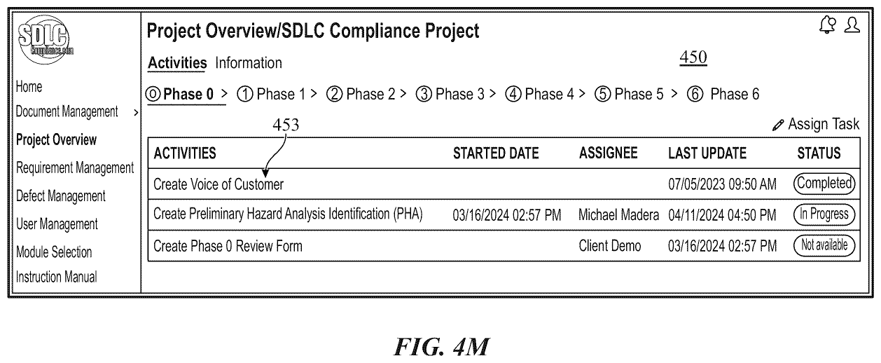

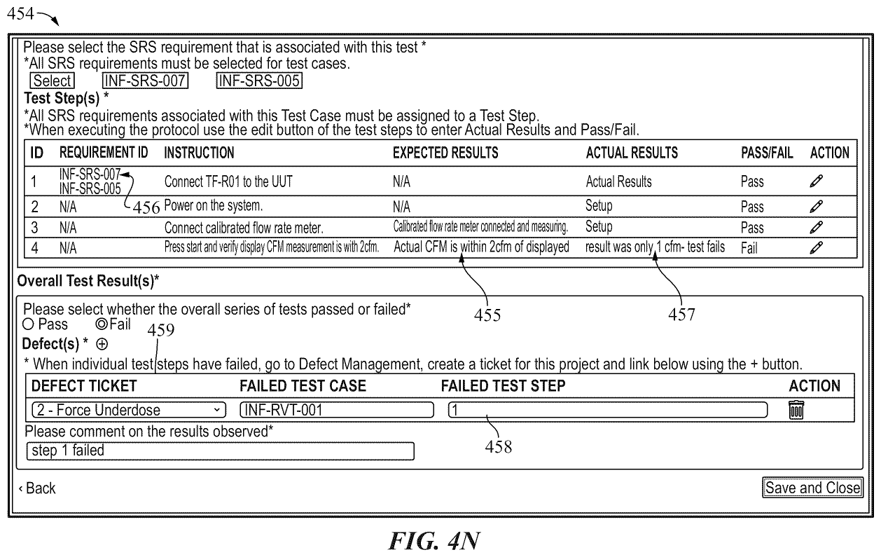

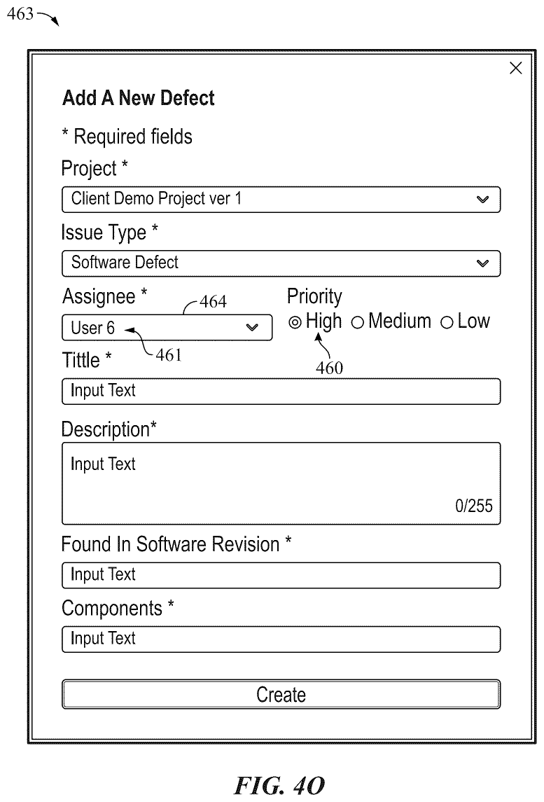

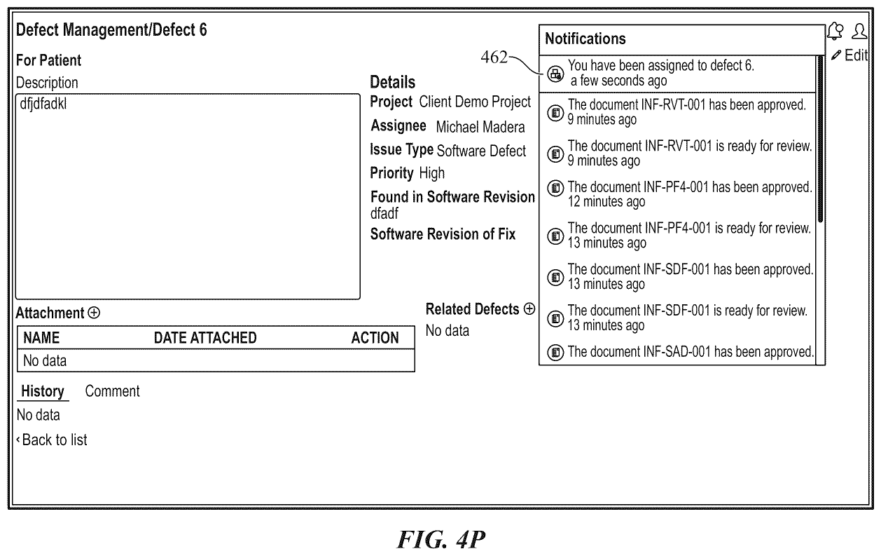

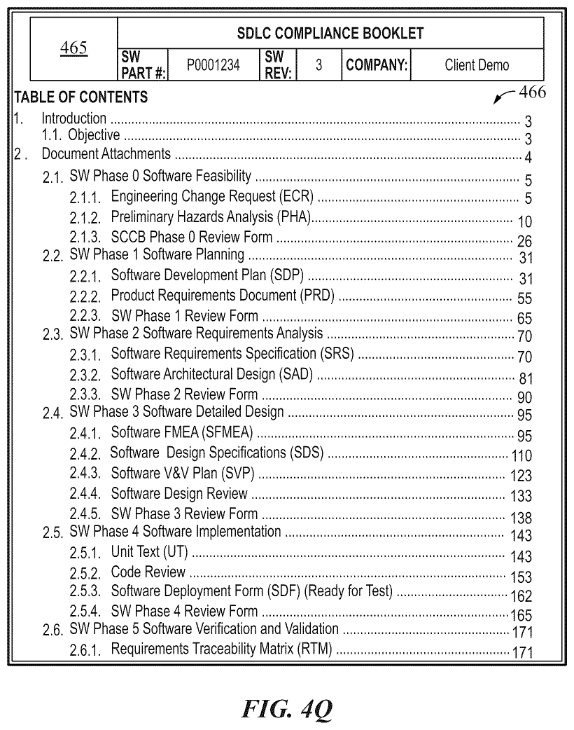

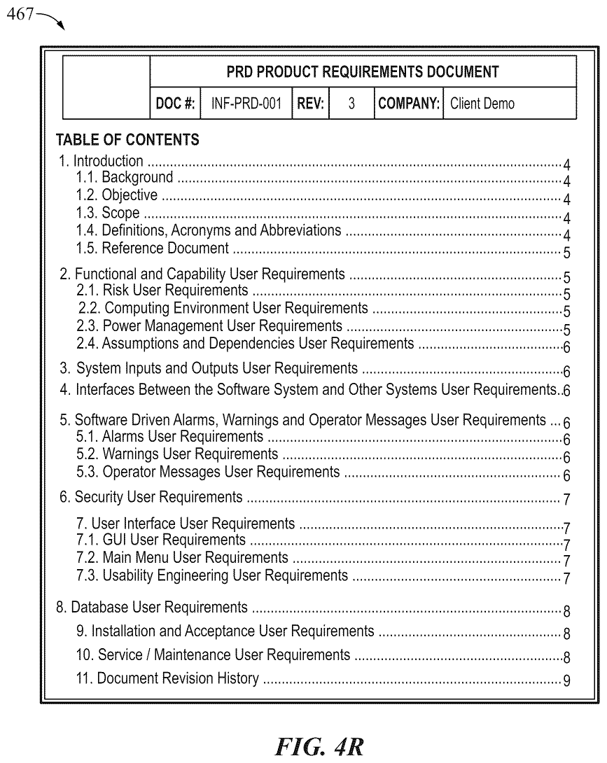

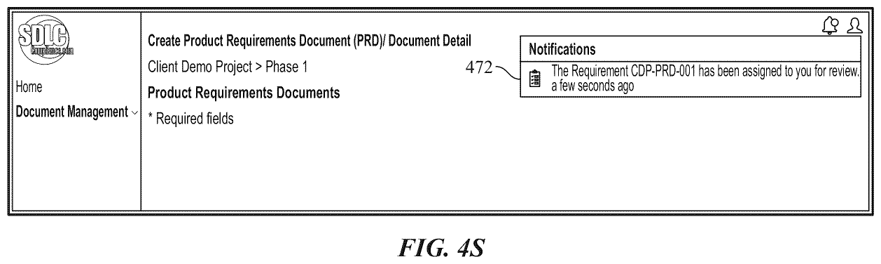

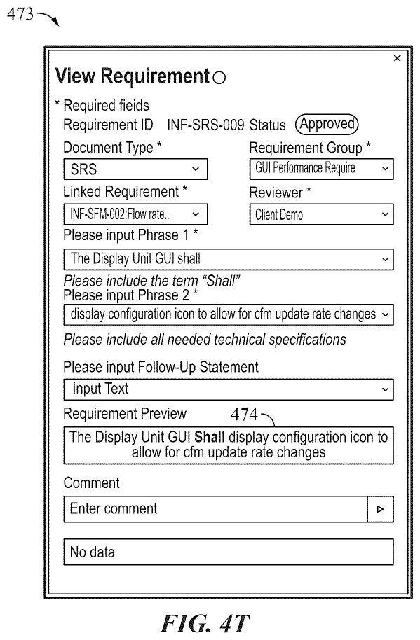

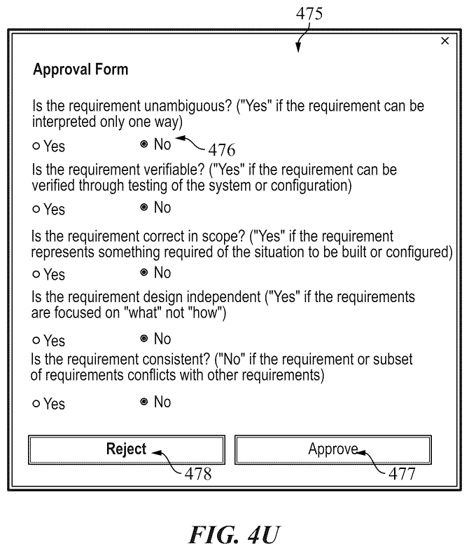

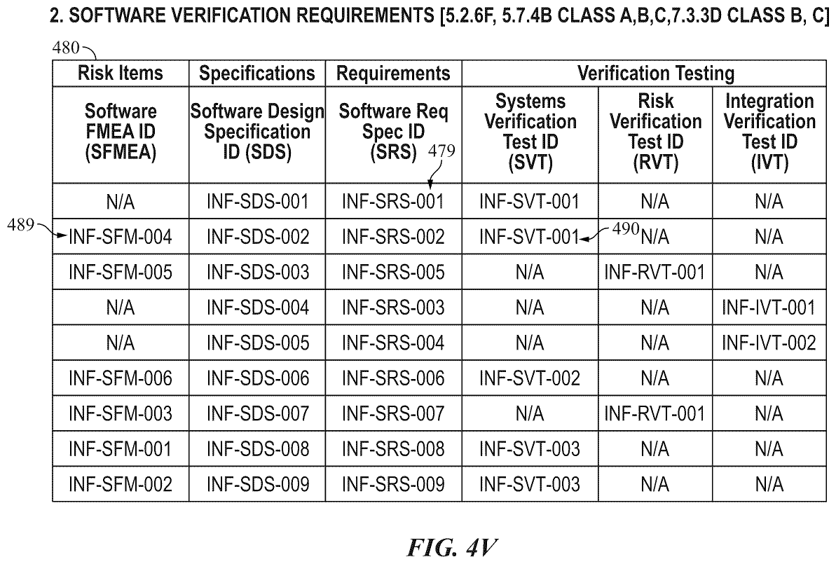

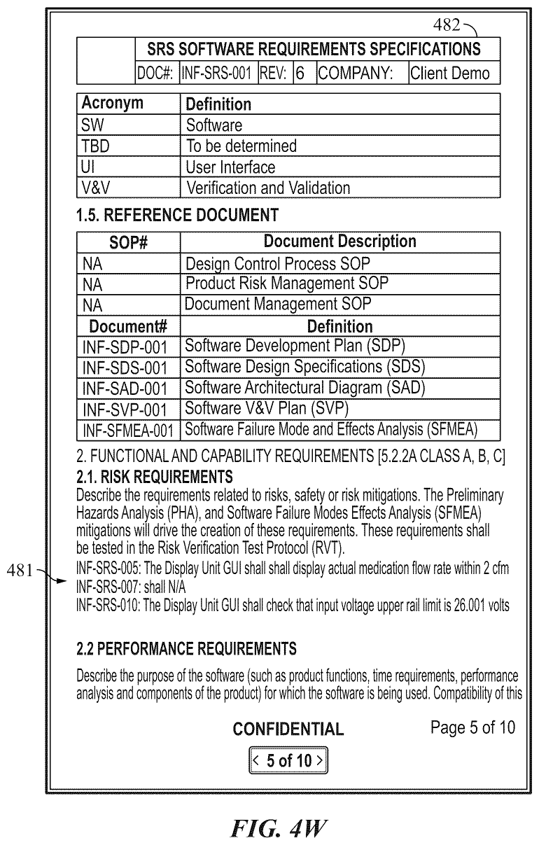

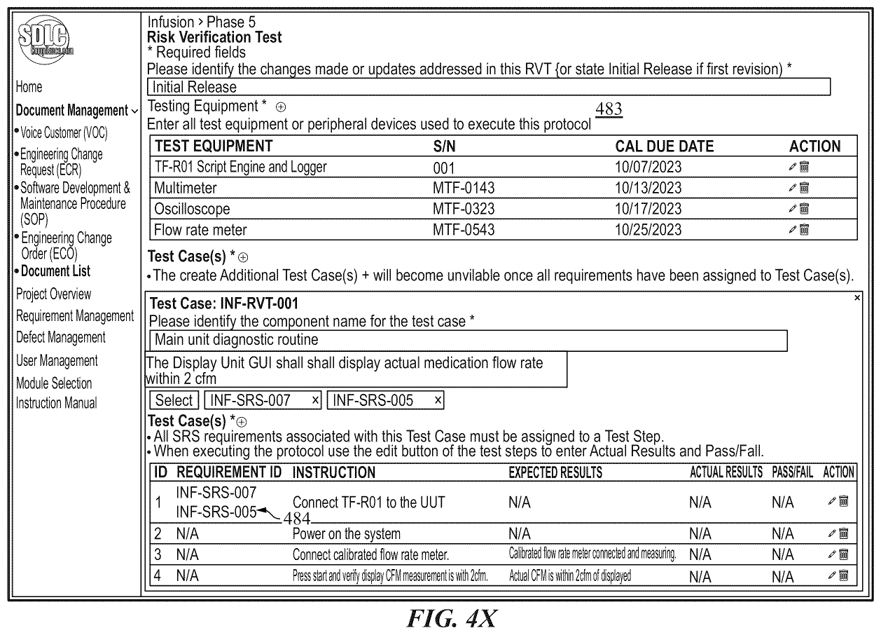

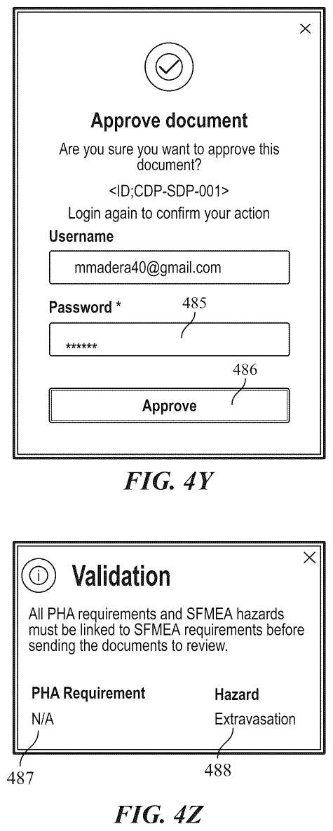

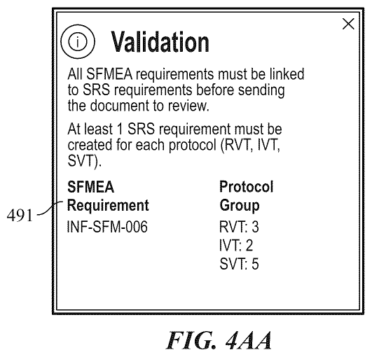

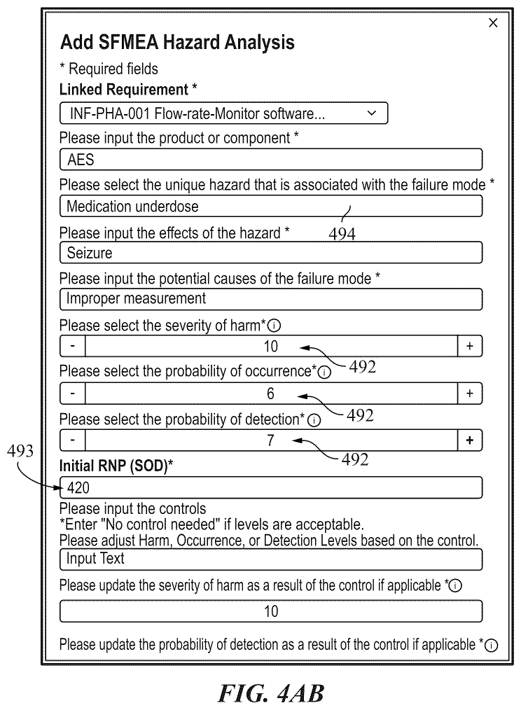

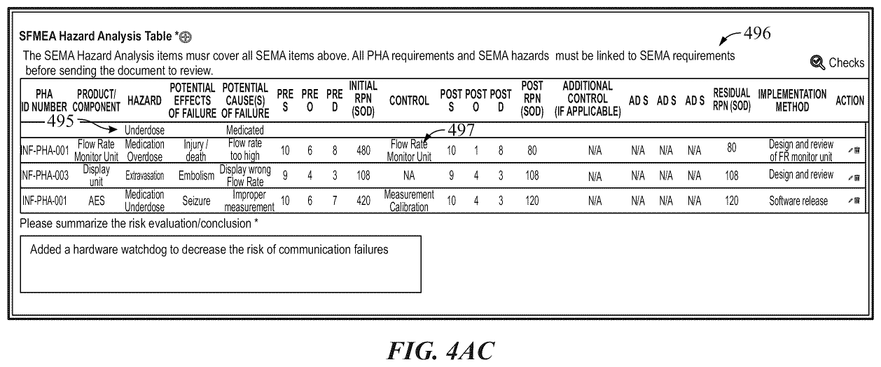

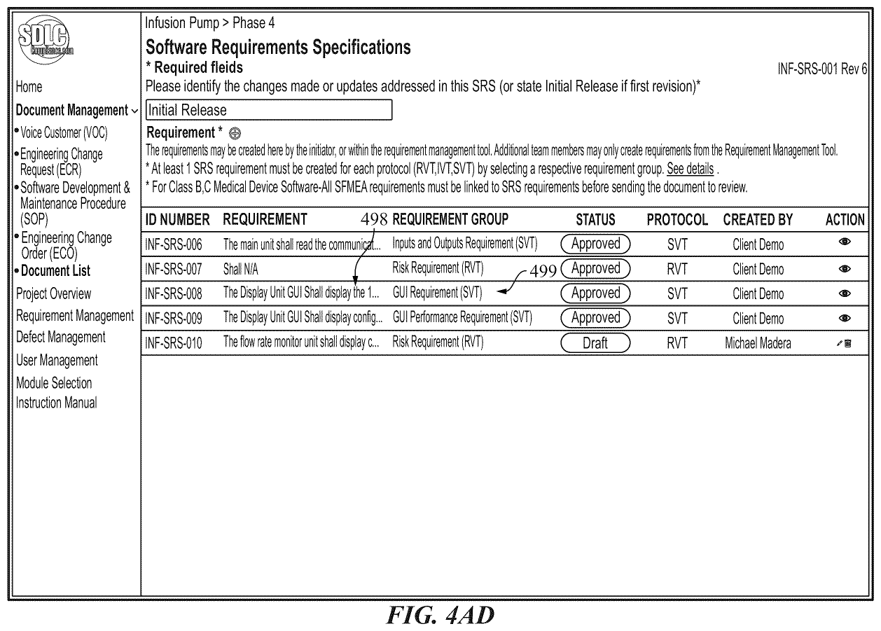

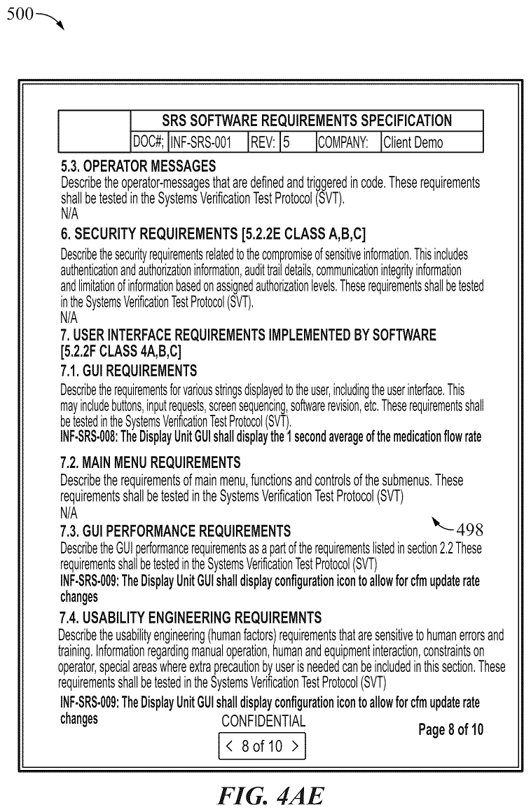

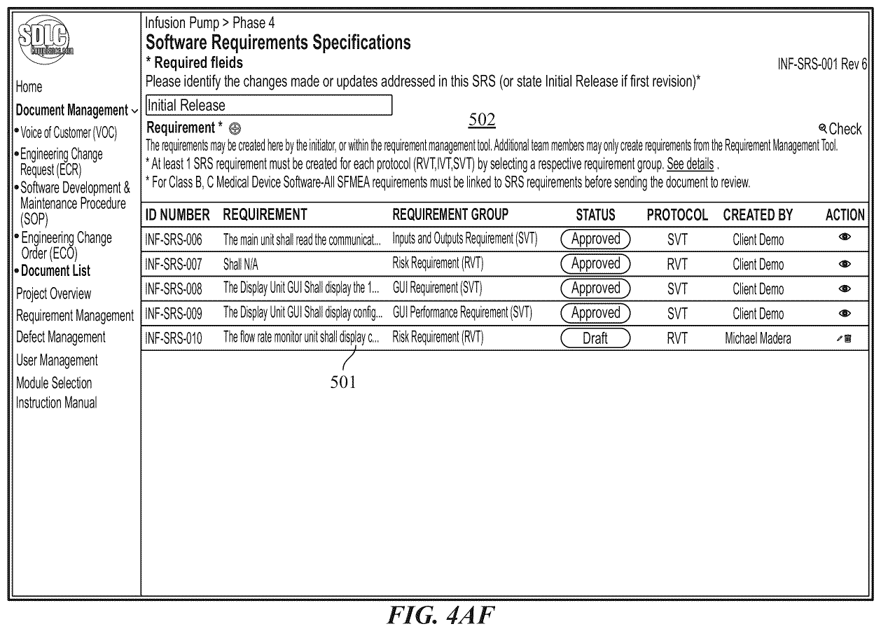

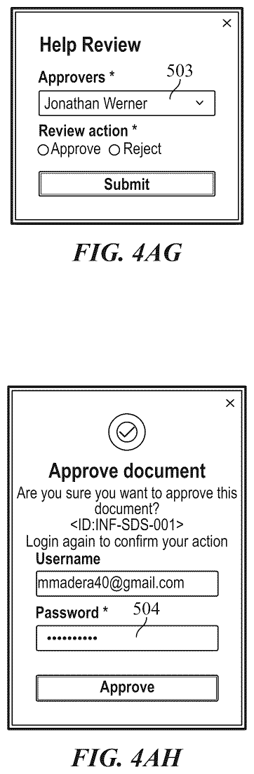

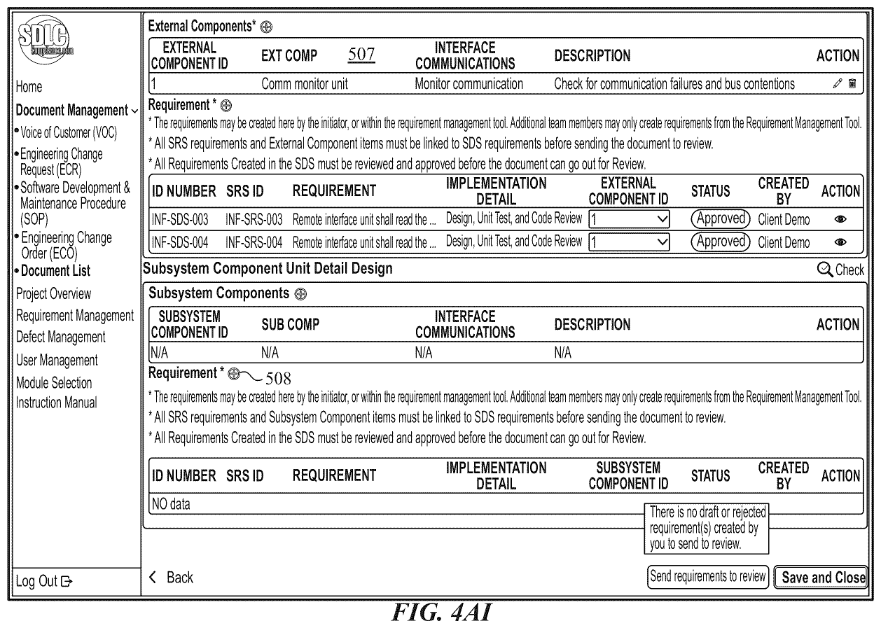

Referring to FIG. 3A, a flowchart of a method 300 for facilitating medical device software development compliance is shown. In some embodiments, the method 300 can receive 310 a request to generate a first project profile for developing a first medical device software application. For example, referring to FIG. 4A, a project name, abbreviation, and an identifier (e.g., voice of customer identifier) can be inputted in a submission form 401 to generate the first project profile.

Referring back to FIG. 3A, in some embodiments, the method 300 can receive 312 role assignment data identifying a first plurality of user accounts with assigned roles. For example, referring to FIG. 4A, a first plurality of user accounts (e.g., user 1, user 2, user 3, user 4, and user 5) can be assigned the roles of a project manager, a product manager, a quality manager, a quality engineer, and a regulatory affairs member using the submission form 401. Upon receiving data inputted in the submission form 401, the method 300 can generate the first project profile.

Referring back to FIG. 3A, in some embodiments, the method 300 can receive 314 a request to generate first documentation associated with the medical device software application. In some embodiments, the method 300 can receive 316 input data such as first input data in response to presenting one or more first questions. For example, referring to FIG. 4B, the first documentation can include preliminary hazard analysis documentation. The method can include receiving first input data 402 in response to presenting one or more first questions 403. Referring to FIGS. 3A and 4B, in some embodiments, the method 300 can receive 316 input data such as second input data indicating one or more first risk controls 404 associated with the medical device software application.

Referring back to FIG. 3A, in some embodiments, the method 300 can determine 318 a software safety classification of the first medical device software application based on the first input data. In some embodiments, the method 300 can, in response to receiving the first input data and the second input data, generate 320 the first documentation including the software safety classification and the first risk controls. For example, referring to FIG. 4C, a flowchart 406 can be included in first documentation 407 to show how input data submitted in response to the one or more first questions 403 is used to generate the software safety classification 405. Referring to FIG. 4D, the first documentation 407 can include a table that includes the first risk controls 404.

Referring back to FIG. 3A, in some embodiments, the method 300 can request 322 approval of the first documentation from each of the first plurality of user accounts with assigned roles. For example, referring to FIG. 4E, approval can be requested by submitting an approval request form 406 and providing authentication information 412 (e.g., username and password). In response to submitting the form, in some embodiments, the method 300 can request approval of the first documentation 407 from each of the first plurality of user accounts with assigned roles. Referring back to FIG. 3A, after each of the first plurality of user accounts with assigned roles approves the first documentation 407, the method 300 can receive 322 the approval of the first documentation from each of the first plurality of user accounts with assigned roles.

In some embodiments, the method 300 can modify 324 the first documentation to indicate the approval from each of the first plurality of user accounts and their assigned roles. For example, referring to FIG. 4F, the first documentation 407 can be modified to include signatures 408 indicating approval from each of the first plurality of user accounts and their assigned roles.

Referring back to FIG. 3A, in some embodiments, the method 300 can store 326 the first documentation in memory (e.g., 204 in FIG. 2). Referring to FIGS. 3A and 4G, the method 300 can include 328 at least a first identifier 409 of the first documentation 407 in a document management dashboard 410 associated with the first project profile of the medical device software application.

Referring to FIGS. 3A and 4H, in some embodiments, the method 300 can generate 330 one or more first requirements (e.g., 417 in FIG. 4I) in a requirements tool based on the one or more first risk controls (e.g., 404 in FIG. 4D) in response to the submission of a requirements form 411. Generally, a requirement is generated to drive design and can be created to mitigate a risk corresponding to a risk control. Referring to FIG. 4I, a requirements dashboard 420 can be generated that includes a plurality of requirements 421, wherein a first approved requirement 423 is associated with a first requirement identifier 422.

Referring to FIGS. 3A and 4J, in some embodiments, the method 300 can generate 332 a plurality of project phases 415 associated with the first project profile based at least on the software safety classification (e.g., 405 in FIG. 4C). The plurality of project phases 415 can be associated with respective sets of tasks 416, wherein one or more tasks of the sets of tasks 416 are generated based at least on the software safety classification and at least a first task of the one or more tasks is associated with the one or more first requirements (e.g., 417 in FIG. 4I). For example, a medical device software application that would not introduce harm to a patient if it failed to operate would result in fewer tasks and/or project phases 415 being generated.

Referring to FIG. 3B, a flowchart of a method 400 for facilitating medical device software development compliance is shown.

In some embodiments, the method 400 can cause 342 a home dashboard to be presented to a first user account of a second plurality of user accounts. Referring to FIG. 4K, a home dashboard 440 can include: one or more project profiles 441 assigned to the first user account; one or more first documentations 442 assigned to the first user account; one or more second documentations 443 created by the first user account; one or more first draft requirements 444 assigned to the first user account; one or more second draft requirements 445 created by the first user account; and one or more statuses 446 of the one or more first documentations 442, the one or more second documentations 443, one or more first draft requirements 444, and one or more second draft requirements 445, and one or more first defect tickets 447 assigned to the first user account; one or more second defect tickets 448 created by the first user account wherein each status of the one or more statuses is an indication of draft, approved, in waiting, open or rejected.

Referring back to FIG. 3B, in some embodiments, the method 400 can generate 344 a projects dashboard. For example, referring to FIG. 4L, a projects dashboard 450 can include a list of project identifiers 451. Referring to FIG. 4M, in response to receiving a selection of a project identifier 452, the method 400 can cause a task identifier 453 of at least one task of the sets of tasks to be presented.

Referring back to FIG. 3B, in some embodiments, the method 400 can generate 346 testing documentation. For example, the method 400 can receive a request to generate first testing documentation associated with the first task. Referring to FIG. 4N, in some embodiments, first testing documentation 454 can include a first requirement identifier 456. The method 400 can receive third input data 457 to be included in the first testing documentation 454, the third input data 457 identifying a first testing action. In some embodiments, the method 400 can determine that the third input data 457 indicates a software defect when, for example, the third input data 457 does not meet expected results 455.

Referring back to FIG. 3B, in some embodiments, the method 400 can generate 348 defect tickets. For example, referring to FIG. 4N, in response to determining that the third input data 457 indicates the software defect, the method 400 can force a first user to generate a defect ticket in response to a test failure associated with the first testing action. For example, the user can be forced to identify the first testing action 458 that resulted in the test failure. In response to determining that the third input data 457 indicates the software defect, the method 400 can prompt and force the first user to generate a first software defect ticket 459. For example, when the user indicates the test failure associated with the first testing action and saves their input data, a defect ticket 459 is generated including the user's input data, and the defect ticket 459 is stored in memory. The method 400 can associate the first software defect ticket 459 with the first testing action 458.

Referring to FIG. 4O, a defect editing page 463 is shown. In some embodiments, the method 400 can receive fourth input data 460 indicating a priority level of the first software defect ticket 459. The method 400 can also receive a request to assign the first software defect ticket to a first user account 461 of a second plurality of user accounts via, for example, a drop down menu 464. Referring to FIG. 4P, after the defect ticket 459 is created, the method 400 can cause a first notification 462 to be presented to the first user account 461, the first notification 462 including a link to the defect editing page 463 for editing the first software defect ticket 459.

Referring to FIGS. 3B and 4Q, in some embodiments, the method 400 can combine 360 all documentation and generate a master table of contents 465 indicating page numbers 466 where documentation is located in the combined documentation. For example, the method 400 can receive a request to combine all documentation associated with the first project profile; in response to receiving the request to combine all documentation associated with the first project profile, the method 400 can generate the combined documentation by combining all documentation associated with the first project profile. In some embodiments, the combined documentation includes the first documentation 407, the first testing documentation 454, and the first software defect ticket 459; and the table of contents 465 including links to each documentation in the combined documentation. Referring to FIG. 4R, in some embodiments, each documentation in the combined documentation includes a respective table of contents such as table of contents 467.

In some embodiments, the method 400 can generate 350 requirements. For example, referring to FIG. 4H, the method 400 can receive a request to generate a new requirement and receive a selection of a standardized prefix 468 to be included in a content of the new requirement. The method 400 can receive third input data 469 to be included in the content of the new requirement, and combine the third input data 469 with the selected standardized (e.g., predetermined or previously stored) prefix 468. The method 400 can include the combined third input data and the selected standardized prefix 470 in the content of the new requirement.

As another example, the third input data 469 can be received as a selection of a standardized (e.g., predetermined) suffix 469 to be included in a content of the new requirement. The method 400 can combine the fourth input data 471 with the selected standardized suffix 469, and the combined fourth input data and the selected standardized suffix 470 can be included in the content of the new requirement.

In some embodiments, the new requirement can be added by any suitable user. For example, the user can be a document author, a requirement author, any other author, or a combination thereof. A requirement can be added within a requirement dashboard 420 as shown in FIG. 4, where an add requirement button 509 can be selected to add a requirement. Additionally or alternatively, a requirement can be added within a document such as in FIG. 4AI, where an add requirement button 508 can be selected to add a requirement.

Referring back to FIG. 3B, in some embodiments, the method 400 can request 354 approval of the new requirement from any user account of a second plurality of user accounts. Referring to FIG. 4S, in some embodiments, requesting approval of the new requirement from a first user account includes: causing a notification 472 to be presented to the first user account; the notification includes a link to a requirement reviewing page 473 shown in FIG. 4T, the requirement reviewing page 473 including the content 474 of the new requirement.

In some embodiments, requesting approval of the new requirement from the first user account includes causing at least the content 474 of the new requirement to be presented to the first user account. Referring to FIG. 4U, in some embodiments, the method 400 can cause a plurality of review questions 475 to be presented to the first user account. In response to receiving fourth input data 476 indicating that a predetermined requirement condition has not been met by the content of the new requirement, the method 400 can force the first user account to disapprove the new requirement. For example, the method 400 can disable an approval button 477 to prevent a user from selecting the approval button 477, thereby preventing the approval of the new requirement. The method 400 can enable a disapproval button 478 to allow the user to disapprove the new requirement.

In some embodiments, when the approval button 477 is enabled and selected, the method 400 can receive the approval of the new requirement from the first user account. In response, and referring to FIG. 4V, the method 400 can include at least a requirement identifier 479 of the new requirement in a requirements traceability matrix 480. Referring to FIG. 4W, the method 400 can include the new requirement 481 in requirements documentation 482. Referring to FIG. 4X, the method 400 can include at least a portion 484 (e.g., an identifier) of the new requirement 481 in the testing documentation 483.

Referring to FIG. 4Y, in some embodiments, requesting approval of any requirement or any documentation from each of the first plurality of user accounts with assigned roles can include prompting each of the of the first plurality of user accounts to provide authentication information 485. After inputting authentication information 485, an approval button 486 can be selected to approve any requirement or any documentation.

Referring to FIG. 3B, in some embodiments, the method 400 can utilize 352 a risk control check tool to ensure that all risk controls are associated with requirements. For example, referring to FIG. 4Z, the method 400 can receive a request to check that all risk controls are associated with respective approved requirements of the one or more approved requirements; in response to receiving the request to check that all risk controls are associated with respective approved requirements, the method 400 can determine that at least one risk control of the one or more first risk controls (e.g., 404 in FIG. 4B) is not associated with an approved requirement of the one or more approved requirements. In response, the method 400 can cause an identifier 487 to be presented, wherein the identifier 487 can be a risk control identifier of the at least one risk control or a requirement identifier 487.

In some embodiments, the method 400 can receive a request to check that all risk controls are associated with respective identified potential software hazards of one or more identified potential software hazards; in response to receiving the request to check that all risk controls are associated with respective identified potential software hazards of the one or more identified potential software hazards, determine that the at least one risk control (e.g., 404 in FIG. 4B) is not associated with any identified potential software hazard of the one or more identified potential software hazards. In response, the method 400 can cause an identifier 488 to be presented, wherein the identifier 488 corresponds to the potential software hazard.

Referring to FIGS. 3B and 4V, in some embodiments, the method 400 can cause 356 a requirements traceability matrix 480 to be presented. The requirements traceability matrix 480 includes: one or more requirement identifiers 479 respectively corresponding to the one or more approved requirements; one or more risk control identifiers 489 of the one or more first risk controls; and one or more test identifiers 490 associated with the first testing documentation.

In some embodiments, the method 400 can provide 358 a link to a requirements dashboard (e.g., 420 in FIG. 4I); in response to receiving a selection of the link to the requirements dashboard 420, the method 400 can cause the requirements dashboard 420 to be presented, the requirements dashboard including: a plurality of requirements 421 including the one or more approved requirements 422; one or more statuses 505 corresponding to respective requirements of the plurality of requirements, the one or more statuses indicating that at least one requirement of the plurality of requirements is in draft, rejected, in waiting, or approved; one or more user assignees 506 respectively corresponding to respective requirements of the plurality of requirements.

Referring back to FIG. 3B, in some embodiments, the method can check 362 that all child requirements are associated with approved parent requirements. For example, referring to FIG. 4AA, the method 400 can receive a request to check that all approved child requirements are associated with respective approved parent requirements of one or more approved parent requirements; in response to receiving the request to check that all approved child requirements are associated with respective approved parent requirements of the one or more approved parent requirements, determine that at least one requirement of the one or more approved requirements is not associated with any parent requirement of the one or more approved parent requirements; causing a requirement identifier 491 of the at least one parent requirement to be presented.

Referring back to FIGS. 3B and 4AB, in some embodiments, the method 400 can determine 364 a combined risk score for a medical device software application. The method 400 can receive first input data 492 indicating one or more risk levels of a potential software hazard. In response, the method 400 can determine a combined risk score 493 based on the one or more risk levels; and color code a hazard identifier 494 of the potential software hazard based on the determined combined risk score 493, or color code the combined risk score 493. Color coding can include including a background color that corresponds to the combined risk score 493.

Referring to FIGS. 3B and 4AC, in some embodiments, the method 400 can receive third input data indicating one or more potential software hazards 495, and generate 366 risk documentation 496 including at least the one or more risk controls 497, and the one or more potential software hazards 495.

Referring to FIG. 3B, in some embodiments, the method can associate 370 any requirement with one or more documentation templates. For example, referring to FIGS. 4AD and 4AE, the method 400 can receive a classification 499 of the first new requirement 498 (e.g., a user interface requirement), and determine that the first new requirement is relevant to one or more documentation templates (e.g., 500 in FIG. 4AE) based on the classification of the first new requirement 498. The method 400 can associate the first new requirement 498 with the one or more documentation templates 500 determined to be relevant to the first new requirement 498 based on the classification 499 of the first new requirement 498.

Referring back to FIG. 3B, in some embodiments, the method 400 can include 372 requirements in new requirements documentation based on the status of requirements. For example, referring to FIG. 4AF, the method 400 can generate new requirements documentation 502, and determine that at least a portion (which can be displayed in full when the mouse cursor is hovered over it) 501 of the requirements are associated with an in draft status or in waiting status; including all requirements of at least the portion 501 of the requirements in new requirements documentation 502.

Referring back to FIG. 3B, in some embodiments, the method 400 can receive 368 approval for any requirement from other user accounts than the user account assigned to the requirement. For example, referring to FIG. 4AG, the method 400 can receive approval from a fifth user account of the second plurality of user accounts, wherein the fifth user account is different from the fourth user account 503 indicated by the fifth user account. Referring to FIG. 4AH, the fifth user account (which can already be logged in) can provide their authentication information 504 to approve on behalf of the fourth user account 503.

All rights including copyrights in the code included herein are vested in and the property of the Applicant. The Applicant retains and reserves all rights in the code included herein, and grants permission to reproduce the material only in connection with reproduction of the granted patent and for no other purpose.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

What is claimed is:

1. A method for facilitating medical device software development compliance, comprising:
receiving role assignment data identifying a first plurality of user accounts with assigned roles;
receiving a request to generate a first project profile for developing a first medical device software application;
receiving a request to generate first documentation associated with the medical device software application;
receiving first input data in response to presenting one or more first questions;
determining a software safety classification of the first medical device software application based on the first input data;
receiving second input data indicating one or more first risk controls associated with the medical device software application;
in response to receiving the first input data and the second input data, generating the first documentation including the software safety classification and the first risk controls;
requesting approval of the first documentation from each of the first plurality of user accounts with assigned roles;
receiving the approval of the first documentation from each of the first plurality of user accounts with assigned roles;
modifying the first documentation to indicate the approval from each of the first plurality of user accounts and their assigned roles;
storing the first documentation in memory;
including at least a first identifier of the first documentation in a document management dashboard associated with the first project profile of the medical device software application;
generating one or more first approved requirements based on the one or more first risk controls, wherein a first approved requirement is associated with a first requirement identifier;
generating a plurality of project phases associated with the first project profile based at least on the software safety classification, wherein:
the plurality of project phases are associated with respective sets of tasks;
one or more tasks of the sets of tasks are generated based at least on the software safety classification;
at least a first task of the one or more tasks is associated with the one or more first approved requirements.

2. The method of claim 1, further comprising:
receiving a request to generate first testing documentation associated with the first task, the first testing documentation including the first requirement identifier;
receiving third input data to be included in the first testing documentation, the third input data identifying a first testing action;
determining that the third input data indicates a software defect;
in response to determining that the third input data indicates the software defect, forcing a first user to indicate a test failure associated with the first testing action;
in response to determining that the third input data indicates the software defect, prompting and forcing the first user to generate a first software defect ticket;
associating the first software defect ticket with the first testing action;
receiving fourth input data indicating a priority level of the first software defect ticket;
receiving a request to assign the first software defect ticket to a first user account of a second plurality of user accounts;
causing a first notification to be presented to the first user account of the second plurality of user accounts, the first notification including a link to a defect editing page for editing the first software defect ticket.

3. The method of claim 2, further comprising:
receiving a request to combine all documentation associated with the first project profile;
in response to receiving the request to combine all documentation associated with the first project profile, generating combined documentation by combining all documentation associated with the first project profile, the combined documentation including:
the first documentation, the first testing documentation, and the first software defect ticket; and
a table of contents including links to each documentation in the combined documentation;
wherein each documentation in the combined documentation includes a respective table of contents.

4. The method of claim 1, wherein a number of the one or more tasks generated is reduced based at least on the software safety classification.

5. The method of claim 2, further comprising:
causing a requirements traceability matrix to be presented, wherein the requirements traceability matrix includes:
one or more requirement identifiers respectively corresponding to the one or more approved requirements;
one or more risk control identifiers of the one or more first risk controls; and
one or more test identifiers associated with the first testing documentation.

6. The method of claim 1, further comprising:
providing a link to a requirements dashboard;
in response to receiving a selection of the link to the requirements dashboard, causing the requirements dashboard to be presented, the requirements dashboard including:
a plurality of requirements including the one or more approved requirements;
one or more statuses corresponding to respective requirements of the plurality of requirements, the one or more statuses indicating that at least one requirement of the plurality of requirements is in draft, rejected, in waiting, or approved;
one or more user assignees respectively corresponding to respective requirements of the plurality of requirements.

7. The method of claim 1, further comprising:
receiving a request to check that all risk controls are associated with respective approved requirements of the one or more approved requirements;
in response to receiving the request to check that all risk controls are associated with respective approved requirements, determining that at least one risk control of the one or more first risk controls is not associated with an approved requirement of the one or more approved requirements;
receiving a request to check that all risk controls are associated with respective identified potential software hazards of one or more identified potential software hazards;
in response to receiving the request to check that all risk controls are associated with respective identified potential software hazards of the one or more identified potential software hazards, determining that the at least one risk control is not associated with any identified potential software hazard of the one or more identified potential software hazards; causing a risk control identifier of the at least one risk control to be presented.

8. The method of claim 1, further comprising:
generating a projects dashboard including a list of project identifiers;
in response to receiving a selection of a project identifier, causing a task identifier of at least one task of the sets of tasks to be presented.

9. The method of claim 1, further comprising:
receiving a request to generate a new requirement;
receiving a selection of a standardized prefix to be included in a content of the new requirement;
receiving third input data to be included in the content of the new requirement;
combining the third input data with the selected standardized prefix;
including the combined third input data and the selected standardized prefix in the content of the new requirement;
requesting approval of the new requirement from a first user account of a second plurality of user accounts.

10. The method of claim 1, further comprising:
receiving a request to generate a new requirement;
receiving a selection of a standardized suffix to be included in a content of the new requirement;
receiving third input data to be included in the content of the new requirement;
combining the third input data with the selected standardized suffix;
including the combined third input data and the selected standardized suffix in the content of the new requirement;
requesting approval of the new requirement from a first user account of a second plurality of user accounts.

11. The method of claim 9, wherein requesting approval of the new requirement from the first user account includes:
causing a notification to be presented to the first user account; wherein the notification includes a link to a requirement reviewing page, the requirement reviewing page including the content of the new requirement.

12. The method of claim 9, wherein requesting approval of the new requirement from the first user account includes:
causing at least the content of the new requirement to be presented to the first user account;
causing a plurality of review questions to be presented to the first user account;
in response to receiving fourth input data indicating that a predetermined requirement condition has not been met by the content of the new requirement, forcing the first user account to disapprove the new requirement.

13. The method of claim 9, further comprising:
receiving the approval of the new requirement from the first user account;
including at least a requirement identifier of the new requirement in a requirements traceability matrix;
including the new requirement in requirements documentation,
including the new requirement in the testing documentation.

14. The method of claim 1, wherein requesting approval of the first documentation from each of the first plurality of user accounts with assigned roles comprises:
prompting each of the of the first plurality of user accounts to provide authentication information.

15. The method of claim 1, further comprising:
receiving a request to check that all approved child requirements are associated with respective approved parent requirements of one or more approved parent requirements;
in response to receiving the request to check that all approved child requirements are associated with respective approved parent requirements of the one or more approved parent requirements, determining that at least one requirement of the one or more approved requirements is not associated with any parent requirement of the one or more approved parent requirements;
causing a requirement identifier of the at least one parent requirement to be presented.

16. The method of claim 1, further comprising causing a home dashboard to be presented to a first user account of a second plurality of user accounts, the home dashboard including:
one or more project profiles assigned to the first user account;
one or more first documentations assigned to the first user account;
one or more second documentations created by the first user account;
one or more first draft requirements assigned to the first user account;
one or more first draft requirements created by the first user account; and
one or more statuses of the one or more first documentations, the one or more second documentations, one or more first draft requirements assigned to the first user account, and one or more first draft requirements created by the first user account, and
one or more first defect tickets assigned to the first user account;
one or more second defect tickets created by the first user account
wherein each status of the one or more statuses is an indication of draft, approved, or rejected.

17. The method of claim 1, further comprising:
receiving third input data indicating one or more risk levels of a potential software hazard;
determining a combined risk score based on the one or more risk levels; and
color coding a hazard identifier of the potential software hazard based on the determined combined risk score.

18. The method of claim 1, further comprising:
receiving third input data indicating one or more potential software hazards;
generating risk documentation including at least the one or more risk controls, and the one or more potential software hazards.

19. The method of claim 1, further comprising:
receiving, from a first user account of a first plurality of user accounts, the first user account being a document author and a requirement author, and in a requirements tool, a request to generate a first new requirement;
receiving, from the first user account, third input data to be included in content of the first new requirement;
including the third input data in the content of the first new requirement;
receiving, from a second user account of the second plurality of user accounts, the second user account being a requirement author, a request to generate a second new requirement in requirements documentation;
receiving, from the second user account, fourth input data to be included in content of the second new requirement;
including the fourth input data in the content of the second new requirement;
requesting approval of the first new requirement from a third user account of the second plurality of user accounts;
requesting approval of the second new requirement from a fourth user account of the second plurality of user accounts.

20. The method of claim 19, further comprising:
receiving approval from a fifth user account of the second plurality of user accounts, wherein the fifth user account is different from the fourth user account;

21. The method of claim 19, further comprising:
receiving a classification of the first new requirement;
determining that the first new requirement is relevant to one or more documentation templates based on the classification of the first new requirement;
associating the first new requirement with the one or more documentation templates determined to be relevant to the first new requirement based on the classification of the first new requirement.

22. The method of claim 1, further comprising:
generating new requirements documentation;
determining that at least a portion of the requirements are associated with an in draft status or in waiting status;
including all requirements of at least the portion of the requirements in new requirements documentation.
